(19) United States
(12) Patent Application Publication
(10) Pub. No.: US 2016/0340593 A1
(43) Pub. Date: Nov. 24, 2016

(54) TANDEM PHOTOCHEMICAL-THERMOCHEMICAL PROCESS FOR HYDROCARBON PRODUCTION FROM CARBON DIOXIDE FEEDSTOCK

(71) Applicant: THE BOARD OF REGENTS OF THE UNIVERSITY OF TEXAS SYSTEM, Austin, TX (US)

(72) Inventors: Frederick Macdonnell, Arlington, TX (US); Brian Dennis, Arlington, TX (US); Wilaiwan Chanmanee, Arlington, TX (US)

(21) Appl. No.: 15/111,636

(22) PCT Filed: Jan. 16, 2015
(86) PCT No.: PCT/US15/11800

§ 371 Date: Jul. 14, 2016

Related U.S. Application Data
(60) Provisional application No. 61/928,719, filed on Jan. 17, 2014.

(51) Int. Cl. C10G 2/00; C07C 41/01; C07C 29/159; B01J 23/75; B01J 35/00

(52) U.S. Cl. CPC C10G 2/40; C10G 2/00; B01J 23/75; B01J 35/004; C07C 29/159; C07C 41/01

(57) ABSTRACT

The present invention is directed at an improved process for generating heavier hydrocarbons from carbon dioxide and/or carbon monoxide and water using tandem photochemical-thermochemical catalysis in a single reactor. Catalysts of the present disclosure can comprise photoactive material and deposits of conductive material interspersed on the surface thereof. The conductive material can comprise Fischer-Tropsch type catalysts.

This application claims priority to U.S. Provisional Application No. 61/928,719 filed Jan. 17, 2014. The entire text the above-referenced disclosure is specifically incorporated herein by reference without disclaimer.

### BACKGROUND OF THE INVENTION

A. Field of the Invention

The invention generally concerns thermal, photocatalytic processes and systems that can be used to produce hydrocarbons from water and C₁ feedstocks, e.g., CO and/or CO₂.

B. Description of Related Art

Recycling CO₂ to produce hydrocarbons, particularly long chain hydrocarbons, in a commercially viable manner has long been a goal of scientific research. Such a process could produce a chemical fuel and assist in curbing the effect of climate change.

In order to achieve commercial viability, the energy required must be provided from a renewable source. One source that holds particular promise is the sun. Solar light energy provides a seemingly infinite source of energy. Thus, harvesting the energy of solar light and its subsequent storage in the form of chemical fuels hold promise to address the current and future demand of energy supply.

Despite nearly 40 years of research on the photocatalytic reduction of CO₂, the scientific community is still a long way from efficient and commercially viable devices. Presently, yields are too low to be viable and predominantly produce methane. The highest rates of product formation generally do not exceed tens of μmol of product per hour of illumination per gram of photocatalyst. Habisreutinger et al., “Photocatalytic Reduction of CO₂ on TiO₂ and Other Semiconductors,” 52 Agnew. Chem. Int. Ed. 7372, 7373 (2013). Longer chain hydrocarbons are produced at even lower concentrations. See e.g., Varghese et al., “High-Rate Solar Photocatalytic Conversion of CO₂ and Water Vapor to Hydrocarbon Fuels,” Nano Letters, vol. 9, no. 2, 2009, at p. 734.

### SUMMARY OF THE INVENTION

The present application is directed to compositions, devices, systems, and methods that generate heavier hydrocarbons (i.e., hydrocarbons having ≧2 carbons) by way of coupling the photo-oxidation of water and the photo-reduction of CO or CO₂ with thermal-chemical carbon-chain formation. The energy for which can be largely if not entirely provided by the sun through the use of concentrated solar radiation. Harnessing the sun's energy for the photochemical excitation of a photoactive material as well as the heat needed to favor carbon-chain formation reactions make the described processes energy efficient.

In particular, the present application involves a continuous gas phase process for the photochemical water oxidation under conditions that favor the transfer of the associated electrons and/or protons to drive the reduction of CO₂ or CO and the conversion of the reduced CO or CO₂ products to longer carbon-chain products. Some of these conversion reactions involve Fischer-Tropsch processes that are thermal and pressure driven processes. In addition, the presence of alkylbenzene products suggests that surface bound alkynes are also formed and cyclotrimerize as another method of forming higher carbon number hydrocarbons.

One aspect of the disclosure relates to a solid catalyst comprising a photoactive material support having a surface and a conductive material interspersed on the surface of the support. In various embodiment, the conductive material comprises a metal, e.g., at least one of Co, Fe, and Ru. In various embodiments, the photoactive material support comprises titanium dioxide. In various embodiments, the conductive material is Co. In various embodiments, the catalyst further comprises a hygroscopic additive. For example, the hygroscopic additive can be a salt comprising at least one of the following anions: PO₄³⁻, HPO₄²⁻, H₂PO₄⁻, SO₄²⁻, HSO₄⁻, CO₃²⁻, OH⁻, F⁻, Cl⁻, Br⁻ and I⁻ and at least one of the following cations: Li⁺, Na⁺, K⁺, Rb⁺, Cs⁺, Be²⁺, Mg²⁺, Ca²⁺, Sr²⁺, Ba²⁺ and Al³⁺. In various embodiments, the hygroscopic additive comprises an acid and wherein the acid comprises at least one of the following: H₂SO₄, H₃PO₄, HF, HCl, HBr, and HI. In various embodiments, the hygroscopic additive is disposed on the surface of the photoactive material support. In various embodiments, the catalyst further comprises a redox-active additive. In various embodiments, the redox-active additive comprises a salt comprising at least one of the following cations: Mn²⁺, Mn³⁺, Mn⁴⁺, Fe²⁺, Fe³⁺, Co²⁺, Co³⁺, Ni²⁺ and at least one of the following anions: PO₄³⁻, HPO₄²⁻, H₂PO₄⁻, SO₄²⁻, HSO₄⁻, CO₃²⁻, OH⁻, F⁻, Cl⁻, Br⁻ and I⁻. In various embodiments, the redox-active additive is disposed on the surface of the photoactive material support. In various embodiments, the solid catalyst is a plurality of nanoparticles. In various embodiments, the solid catalyst is coated on a substrate. In various embodiments, the substrate is a surface of a pellet, wherein the pellet is optically transparent. In various embodiments, the pellet is thermally conductive.

A further aspect of the disclosure comprise an apparatus for carrying out thermocatalytic and photocatalytic reactions comprising a reaction vessel having a vessel wall defining a chamber and having a gas inlet and a gas outlet in fluid communication with the chamber, the reaction vessel configured to operate at temperatures greater than 100° C. and to permit electromagnetic radiation to pass through at least a section of the vessel wall and into the chamber and a catalytic body comprising a surface and disposed in the chamber, where disposed on the surface of the catalytic body is the above described solid catalyst.

Relatedly, another aspect relates to a method of coupling photochemical water oxidation with CO₂ or CO reduction and thermochemical carbon-chain formation comprising providing a flow of water and at least one of CO₂ and CO into a reaction chamber containing a supported metal catalyst in accordance with the present disclosure; heating the reaction chamber to a reaction temperature greater than 100° C.; and exposing the supported metal catalyst to electromagnetic radiation, thereby causing photochemical water oxidation, CO₂ or CO reduction, and thermochemical hydrocarbon formation, wherein the hydrocarbons comprise alkanes and alcohols having at least 2 carbons.

Similarly, another aspect of the disclosure relates to a method of converting a gaseous mixture comprising CO₂ and water to hydrocarbons, the method comprising: providing a flow of water and at least one of CO and CO₂ into a reaction chamber containing a supported metal catalyst; heating the reaction chamber to a reaction temperature greater than 100° C.; and exposing the supported metal catalyst to electromagnetic radiation, thereby causing a reaction that generates hydrocarbons from the provided flow, wherein the supported metal catalyst comprises a photoactive material support and a plurality of conductive particles disposed on the support. In various embodiments, the reaction temperature is between 100° C. and 300° C. In various embodiments, the reaction temperature is between 150° C. and 250° C. In various embodiments, heating the reaction chamber comprises directing sunlight reflecting from a solar concentrator onto the reaction chamber. In various embodiments, the photoactive material support is a semiconductor support and the supported metal catalyst is the semiconductor support having a surface with metal particles interspersed on the surface. In various embodiments, the method further comprises collecting the hydrocarbons. In various embodiments, collecting the hydrocarbons comprises passing outflow from the reaction chamber through a separation device comprising at least one of a condensation column, an adsorbent material, membrane, or centrifuge. In various embodiments, the method further comprises recycling the outflow from the separation device into the reaction chamber. In various embodiments, the hydrocarbons include alkanes or alcohols having at least 2 carbons. In various embodiments, the hydrocarbons include alkanes or alcohols having at least 6 carbons. In various embodiments, the hydrocarbons include at least one of methane, ethane, propane, butane, hexane, heptane, septane, octane, nonane, decane, methanol, ethanol, propanol, butanol, acetone, acetic acid, and alkylbenzene derivatives and oxygenates thereof. In various embodiments, the supported metal catalyst is adapted to absorb electromagnetic radiation having wavelength between 200 nm and 700 nm, between 200 nm and 600 nm, between 200 nm and 500 nm, or between 200 nm and 400 nm.

Another aspect of the disclosure relates to an apparatus for carrying out thermocatalytic and photocatalytic reactions can comprise a reaction vessel having a vessel wall defining a chamber and having a gas inlet and a gas outlet in fluid communication with the chamber, a packed bed comprising a surface and disposed in the chamber, where disposed on the surface of the packed bed is a supported metal catalyst comprising a photoactive material support and a conductive material interspersed on the support; and a gaseous mixture consisting essentially of water and at least one of CO and CO₂ within the chamber at a temperature greater than 100° C. The reaction vessel is configured to operate at temperatures greater than 100° C. and to permit electromagnetic radiation to pass through at least a section of the vessel wall and into the chamber.

Yet another aspect of the disclosure relates to a solar concentrating system comprising an optical concentrating device and a packed bed reactor configured to receive light from the optical concentrating device; a gasification unit in fluid communication with the reaction chamber configured to convert liquid water to steam; and a CO₂ supply line in fluid communication with the reaction chamber. The reactor can comprise a reaction vessel having a vessel wall defining a chamber and having a gas inlet having an inflow and a gas outlet having an outflow, both being in fluid communication with the chamber. The reaction vessel can be configured to operate at temperatures greater than 100° C. and to permit electromagnetic radiation to pass through at least a section of the vessel wall and into the chamber to a packed bed comprising a surface. Disposed on the surface of the packed bed is a supported metal catalyst comprising a photoactive material support and a conductive material interspersed on the support. In various embodiments, the system further comprises a separation unit for extracting hydrocarbons from the outflow. In various embodiments, the system further comprises a gas mixer to mix the steam with carbon dioxide. In various embodiments, the system further comprises a heat exchanger configured to transfer thermal energy from the reaction vessel to the gasification unit.

Yet another aspect of the disclosure relates to a method for concentrating solar radiation to provide light for the photochemical excitation of a supported metal catalyst and to provide the thermal energy needed for carbon-chain formation reactions, the method comprising: providing a flow of water and at least one of CO₂ and CO into a reaction chamber containing a supported metal catalyst comprising a semiconductor, wherein the pressure in the reaction chamber is between 1 atm and 15 atm; and concentrating and directing solar radiation to the reaction chamber, thereby heating the reaction chamber to a reaction temperature greater than 100° C. and causing the photochemical excitation of the semiconductor, wherein hydrocarbons having at least 2 carbons are formed in the reaction chamber. In various embodiments, the supported metal catalyst is a solid catalyst in accordance with the present disclosure. In various embodiments, the flow further comprises water vapor. In various embodiments, some heat from the reaction chamber is transferred to a vaporization unit containing water.

The term “intersperse” is defined as a random or patterned distribution of substantially discrete things, e.g., particles, on the surface of and/or within a medium.

The term “coupled” is defined as connected, although not necessarily directly, and not necessarily mechanically.

The terms “a” and “an” are defined as one or more unless this disclosure explicitly requires otherwise.

The preposition “between,” when used to define a range of values (e.g., between x and y) means that the range includes the end points (e.g., x and y) of the given range and of course, the values between the end points.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the particles, devices, methods, and systems of the present invention that “comprises,” “has,” “includes” or “contains” one or more elements possesses those one or more elements, but is not limited to possessing only those one or more elements. Likewise, an element of a particle, device, method, or system of the present invention that “comprises,” “has,” “includes” or “contains” one or more features possesses those one or more features, but is not limited to possessing only those one or more features.

Furthermore, a structure that is capable performing a function or that is configured in a certain way is capable or configured in at least that way, but may also be capable or configured in ways that are not listed.

The feature or features of one embodiment may be applied to other embodiments, even though not described or illustrated, unless expressly prohibited by this disclosure or the nature of the embodiments.

Any composition, device, method, or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 illustrates a schematic example of the photo-induced formation of an electron-hole pair of the photoactive catalyst composite facilitating the oxidation and reduction reactions. “A” represents an electron acceptor and “D” represents an electron donor.

FIG. 2A illustrates a schematic of photoactive catalyst composite in accordance with the present disclosure.

FIG. 2B illustrates a schematic of a photoactive catalyst composite disposed on a substrate composite in accordance with the present disclosure.

FIG. 3A illustrates a schematic of a reactor in accordance with the present disclosure.

FIG. 3B illustrates a schematic of a reactor in accordance with the present disclosure.

FIG. 4A illustrates a schematic of a system for converting C₁ feedstock and water into hydrocarbons.

FIG. 4B illustrates an array of solar concentrators and reaction vessels in accordance with the present disclosure.

### DETAILED DESCRIPTION OF THE INVENTION

Various features and advantageous details are explained more fully with reference to the non-limiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements will become apparent to those of ordinary skill in the art from this disclosure.

In the following description, numerous specific details are provided to provide a thorough understanding of the disclosed embodiments. One of ordinary skill in the relevant art will recognize, however, that the invention may be practiced without one or more of the specific details, or with other systems, methods, components, materials, and so forth. In other instances, well-known structures, materials, or operations are not shown or described in detail to avoid obscuring aspects of the invention.

The present invention is predicated upon the unexpected realization of a substantially improved process and system for generating heavier hydrocarbons from a C₁ feedstock and water according to a process that generates the required activation energies mostly if not entirely from sunlight. While not wishing to be bound by any particular theory, the present invention is directed at an improved process for generating heavier hydrocarbons from C1 feedstock and water using photocatalytic and Fischer-Tropsch type processes in a single reactor. The improved process can generate heavier hydrocarbons with the use of renewable energy sources. (It should be realized, however, the invention contemplates the optional use of features that provide energy from nonrenewable sources.) Among the advantages, hydrocarbons can be produced at yields greater than 100 μg/g of catalyst per hour and even greater than 200 μg/g of catalyst per hour. In addition, the percentage of heavier hydrocarbons is greater than the percentage of methane and/or methanol.

As described in detail below, the present disclosure contemplates that one or more photochemical reactions and thermal reactions take place in tandem, preferably within a single reaction chamber or single zone within a reaction vessel. Moreover, the photochemical reactions take place at the relatively high temperatures and/or the relatively high pressures needed to facilitate the thermal reactions that produce heavier hydrocarbons at yields greater than 3 μmol/g of catalyst per hour. Preferably, the reaction chamber is maintained so that the C₁ feedstock and water therein are at a temperature greater than or equal to 100° C., and more preferably higher than 120° C. The reaction chamber may exhaust into a recovery unit wherein the generated hydrocarbons are extracted from the exhausted gas stream, and a return path from the recovery unit may couple to the reaction chamber to form a closed loop system, as described herein.

The catalyst of the present disclosure is a composite material preferably in the form of particles that are sufficiently small to be characterized as nanoparticles (e.g., they have an average diameter less than about 100 nm). The catalyst composite comprises a photoactive material and a conductive species (e.g., a supported metal catalyst) on which (not wishing to be bound by a particular theory) water oxidation, C₁ feedstock reduction, and Fischer-Tropsch type reactions are believed to occur causing a gaseous mixture of C₁ feedstock and water, exposed to both sunlight and thermal energy, to generate hydrocarbons, a majority portion of which are heavier hydrocarbons. C₁ feedstock are simple carbon-containing substrates that contain one carbon atom per molecule and include, e.g., methane, carbon dioxide, carbon monoxide, and methanol. In various embodiments, the gas stream comprises C₁ feedstock that is substantially CO and CO₂. In various embodiments, the gas stream comprises C₁ feedstock that is substantially CO or CO₂.

### A. PHOTOACTIVE CATALYSTS

In accordance with the present disclosure, the photoactive catalysts can comprise a photoactive material and a conductive species disposed or interspersed on at least a portion of the surface of the photoactive material. With respect to the photoactive material, it can comprise any material that provides suitable band gap excitations (e.g., semiconductive materials). With respect to the conductive species, it can comprise any material that accepts the photo-generated electrons and facilitates transporting such electrons to the surface for participation in the reduction process and carbon-chain formation. In various embodiments, the photoactive catalyst is a supported metal catalyst.

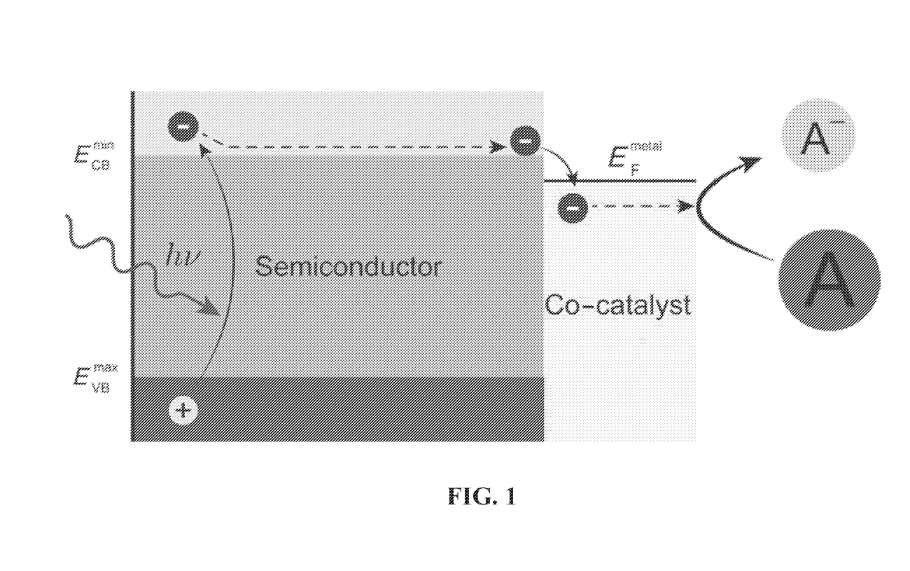

While not wishing to be bound by a particularly theory, with reference to FIG. 1, in various embodiments, the semiconductor(s) is selected to have a band gap that spans the range of the reduction and oxidation potentials relevant to the photo-catalyzed reactions, namely the oxidation of water (≧0.82 V vs NHE at pH 7.0) (1) and the reduction of C₁ feedstock (≦−0.41 V vs NHE at pH 7.0) (2), the later predominantly occurring on the conductive material deposits. For example, the band gap of titanium dioxide is 3.0 and 3.2 eV for rutile and anatase, respectively, and thus, only radiation shorter than 400 nm is absorbed, which is not very matched with the majority of the solar spectrum reaching the earth's surface. The valence band edges for rutile and anatase are well in excess of 0.82 V and the conduction band edge is approximately −0.40 V) In certain embodiments, other semiconductor(s) are selected so that the photoactive catalyst absorbs a wide spectrum of solar radiation. For example, BiVO₄ is a semiconducting metal oxide which absorbs light at wavelengths less than 550 nm and which could be used as a photoactive support for the metal co-catalyst to drive the desired reaction utilizing a greater portion of the solar spectrum. In certain embodiments, the supported metal catalyst is adapted to absorb electromagnetic radiation having wavelength less than 700 nm, less than 600 nm, or less than 500 nm.

Combined with the semiconductive material, conductive materials can comprise a material, such as a metal or metal oxide, that facilitates transporting the photo-generated electrons from the semiconductive material to the surface for reduction of C₁ feedstock (2) and subsequent carbon-chain formation (3). While not wishing to be bound by any particular theory, it is believed that the semiconductor serves as the photo-anode, oxidizing water and transferring electrons and protons to the conductive material islands. Presumably, these form surface hydrides that are the reducing agents for C₁ reduction and subsequent carbon-chain formation reaction.

The oxidation and reduction reactions are summarized below with an example of reaction conditions. With the use of the described photoactive catalyst and methods of the present disclosure, reactions (1)-(3) can take place in a single reactor.

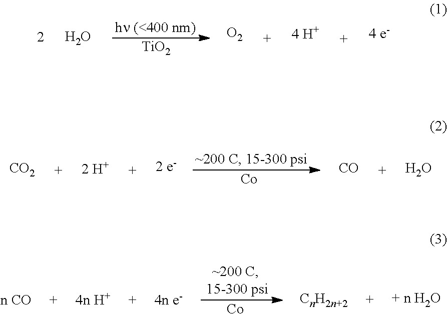

It is noted, particularly where the C1 feedstock includes CO, a series of thermochemical reactions are possible (e.g., reverse water-gas shift chemistry coupled with Fischer-Tropsch chemistry), and could also yield hydrocarbons. To the extent such reactions are occurring, it would be in addition to the coupled photo-thermochemical process described above.

Semi-conductive materials can comprise metal oxides, preferably TiO₂. The TiO₂ can be in any form such as anatase or rutile. Other examples of semi-conductive materials include CdS, TaON, ZnO, and BiVO₄.

In some embodiments, the semi-conductive material is a nanoparticle. The nanoparticle can comprise any shape. The term nanoparticles, refers to a particle having an average width of less than about 200 nm. These nanoparticles may be spherical or close to spherical in shape. Nanoparticles can have a smooth surface or a rough surface, e.g., a highly varied surface with cracks, pits, pores, undulations, or the like to increase the overall surface area. Nanoparticles that are in the form of nanowires, nanotubes, or irregular shaped particles may also be used. Nanoparticles, such as nanotubes, can have a low wall thickness that facilitates transfer of photo-generated charge carriers to the conductive species. If the particles do not have a spherical shape, the size of the particles can be characterized by the diameter of a generally corresponding sphere having the same total volume as the particle. In some embodiments, the nanoparticles have an average diameter of at least 5 nm. In some embodiments, the nanoparticles have an average diameter of less than about 50 nm and even less than about 20 nm.

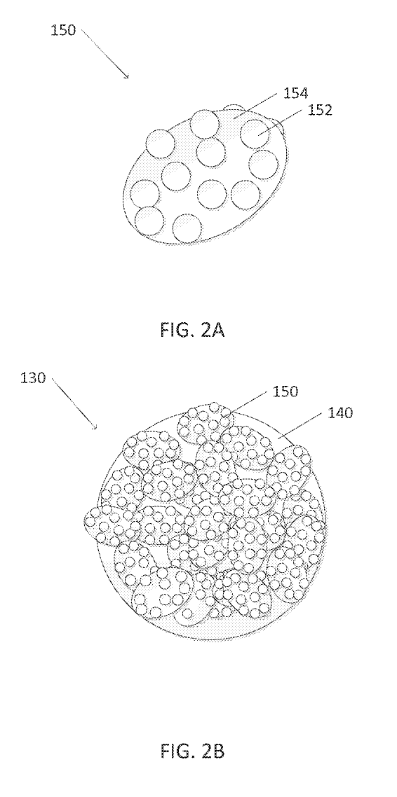

In various embodiments, the conductive material comprises any material suitable as a catalyst in the Fischer-Tropsch reaction. In some embodiments, the conductive material comprises or consists essentially of a metal or metal oxides of the metal selected from the following group: Fe, Co, Ni, Cu, Ru, Rh, Ir, Pd, Pt and Ag or any combination thereof. In some embodiments, the conductive material comprises Co and/or Co₂O₃. In certain embodiments, the conductive material is at least 90%, 91%, 92%, 93%, 94%, 95%, 96%, 97%, 98%, or 99% Co₂O₃. In various embodiments, the conductive material comprises a plurality of small particles, such as metal crystallites or nanoparticles. As schematically illustrated in FIGS. 2A to 2B, the conductive material 152 can be surface decorated or wet-impregnated onto the semi-conductive material 154 such that conductive particles or deposits 152 are disposed or interspersed on the semi-conductive surface 154, referred to together as a metal supported catalyst 150. The % weight of conductive material relative to the semi-conductive material can be any amount between 1% to 30%, such as about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or any value or range there between. In some embodiments, the % weight of the conductive material relative to the semi-conductive material is between about 2% to about 15%.

In some embodiments, the semi-conductive material can comprise a combination of semi-conductive materials and one or more dopants to enhance the efficiency of the catalyst through extension of the absorption range and/or improvement in the charge separation to increase the number of photo-excited electrons and decrease the number that return to the valence band. For example, TiO₂ can be doped with nitrogen, such as nitrogen in the form of ammonium fluoride. The % weight of the dopant relative to the semi-conductive material can be any amount between 0% to 5%, such as between about 1% and 3%.

Alternatively or in addition thereto, a hygroscopic additive can be applied or added to the semiconductor to aid in the stabilization or formation of a surface hydration layer to enhance proton transport during active catalysis. For example, depositing hygroscopic salts or acids onto the semiconductor particles can favor hydration under the process conditions described herein and support proton transport from the sites of water oxidation on the semiconductor surface to the conductor material deposits. Examples of hydroscopic salts include the various salts and acidic salts that can form from combining at least one of the following anions: PO₄³⁻, HPO₄²⁻, H₂PO₄⁻, SO₄²⁻, HSO₄⁻, CO₃²⁻, OH⁻, F⁻, Cl⁻, Br⁻ or I⁻, with at least one of the following Li⁺, Na⁺, K⁺, Rb⁺, Cs⁺, Be²⁺, Mg²⁺, Ca²⁺, Sr²⁺, Ba²⁺, or Al³⁺. Examples of acids include H₂SO₄, H₃PO₄, HF, HCl, HBr and HI. The % weight of the hygroscopic additive relative to the semi-conductive material can be any amount between 0% and 5%, preferably 1% and 3%.

Alternatively or in addition thereto, a redox-active additive could be applied or added to the semiconductor to enhance water oxidation. For example, depositing a redox-active transition-metal salt onto the semiconductor particles can facilitate or enhance the water oxidation process. Examples of the redox active transition metal salts include the various salts that can form from combining at least one of the following cations: Mn²⁺, Mn³⁺, Mn⁴⁺, Fe²⁺, Fe³⁺, Co²⁺, Co³⁺, Ni²⁺, Ru²⁺, Ru³⁺, Ru⁴⁺, Rh⁺, Rh²⁺, Rh³⁺, Ir⁺, Ir²⁺, and Ir³⁺ and at least one of the following anions: PO₄³⁻, HPO₄²⁻, H₂PO₄⁻, SO₄²⁻, HSO₄⁻, CO₃²⁻, O²⁻, OH⁻, F⁻, Cl⁻, Br⁻ and I⁻. The % weight of the redox-active additive relative to the semi-conductive material can be any amount between 0% and 5%, such as between 1% and 3%.

Alternatively or in addition thereto, a supported metal catalyst can be further modified by addition of a basic metal oxide promotor of the Fischer-Tropsch synthesis reaction. For example, the basic metal oxide promotor can comprise an oxide salt comprising at least one of the following cations: Sc³⁺, Y³⁺, La³⁺, Ce³⁺, Pr³⁺, Nd³⁺, Sm³⁺, Eu³⁺, Gd³⁺, Tb³⁺, Dy³⁺, Ho³⁺, Er³⁺, Tm³⁺, Yb³⁺, Ac³⁺, Th³⁺, Pa³⁺, and U³⁺. The % weight of the basic metal oxide promotor relative to the semi-conductive material can be any amount between 0% and 5%, such as between 0.5% and 3%.

In various embodiments, metal supported catalyst 150 is deposited on the surface of a substrate-providing member 140, referred to together as a catalyst body 130. Substrate-providing member 140 can be a molded or extruded body. The surface can be smooth or porous. Substrate-providing member 140 can comprise any suitable material able to withstand the process temperatures and be substantially inert. In various embodiments, the catalyst comprises water soluble components, but is still adapted to withstand the reactant gases and not be significantly dissolved during use. In various embodiments, the material is substantially transparent to visible and ultraviolet light at least within the absorption range of semiconductor. In some embodiment, the material can absorb the infrared radiation received from the sunlight or from the ongoing reaction to facilitate maintaining the high reaction temperatures the reaction chamber, as described below. Examples of material of which substrate-providing member 140 can be composed include glass, quartz, or any other solid UV transmitting medium that is solid at process temperatures, such as temperatures up to 250° C.

Substrate providing member 140 can be any shape for optimizing the surface area upon which catalyst composite 150 is disposed to receive electromagnetic radiation. For example, substrate member 140 can define any shape, e.g., a planar, spherical, ovoidal, elliptical, prismoidal, polyhedron, or pyramidal body. In some embodiments, the catalyst composite 150 can be coated on bead(s), pellet(s), or the like. In other embodiments, catalyst composite 150 can be coated on a body having a generally planar or corrugated surface, such as a fin(s) radially extending out from a central core or a cylindrical body having an outer surface comprising a plurality of undulating or otherwise protruding features to form a corrugated surface. In yet other embodiments, substrate-providing member can comprise three-dimensional substantially porous body or web-like body that provides a substrate and allows sunlight to pass through its full depth.

In addition, substrate providing member 140 can be of any suitable size. For example, when in the shape of a bead, pellet, or particle, substrate providing member 140 can have a minimum width of greater than approximately 1 mm, and can have a maximum width of less than approximately 20 mm. In some embodiments, substrate providing member 140 is substantially spherical, and has a diameter in the range of approximately 1 mm to 10 mm, such as 2 mm, 3 mm, 4 mm, 5 mm, 6 mm, 7 mm, 8 mm, or 9 mm. In other embodiments, when substrate providing member provides a generally planar or corrugated surface or is a porous or web-like body, the dimensions can be such that substrate member 140 extends the length and width of a reaction chamber discussed herein.

The catalyst body 130 can further comprise a medium within which the metal supported catalyst 150 are dispersed. The medium can allow catalyst 150 to adhere to a substrate. In addition, the medium can facilitate surface redox reactions and improve the efficiency of catalyst 150. For example, a medium can comprise an ionomer, e.g., a perfluorosulfonic acid (H⁺ form)/polytetrafluoroethylene copolymer (Nafion®). Other suitable mediums include QPAC (poly(alkylene carbonate)), QPAC 25 (PEC, polyethylene carbonate), QPAC 40 (PPC, polypropylene carbonate), polyvinyl alcohol (PVA), polystyrene-b-poly(ethylene oxide) (PS-b-PEO) polymers, and the like. Other ionomers or guidelines for selecting or designing an ionomer may be found in the following article: Viswanathan & Helen, “Is Nafion, the only choice?”, Bulletin of the Catalysis Society of India, 6 (2007) 50-66, which is hereby incorporated by reference in its entirety. The % weight of a medium relative to the semi-conductive material can be any amount between 0% and 10%, such as 1%, 2%, 3%, 4%, 5%, 6%, 7%, 8%, or 9%.

### B. PHOTOACTIVE CATALYTIC REACTOR

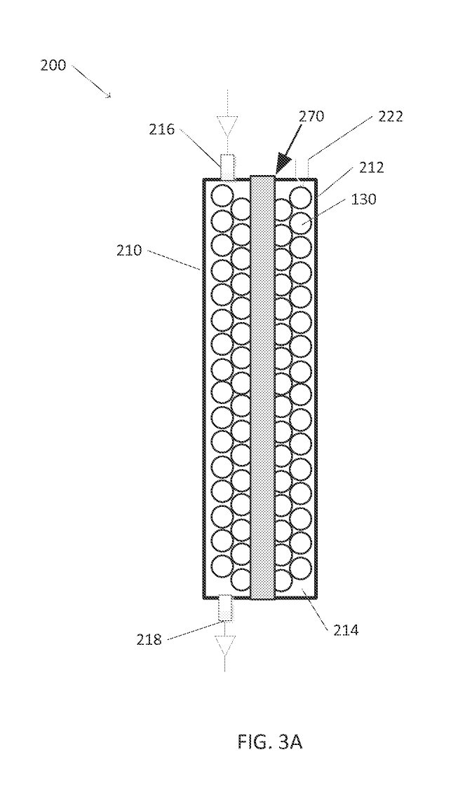

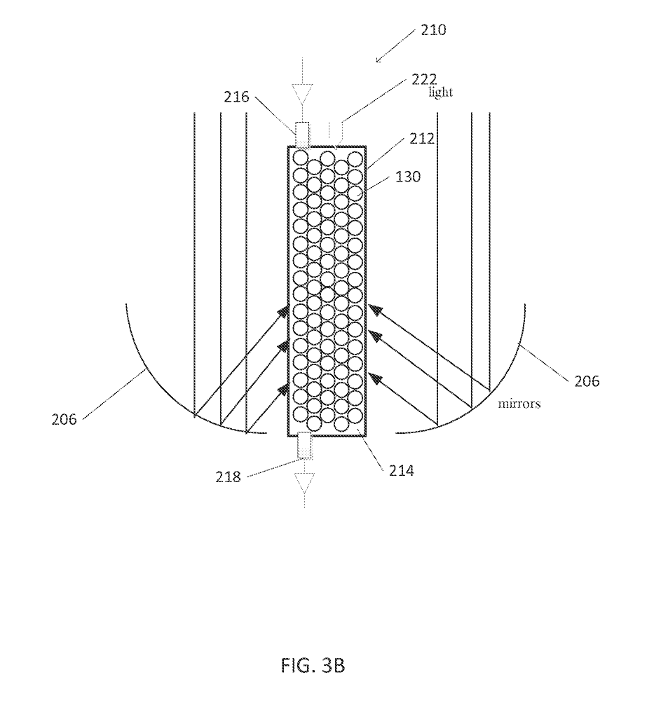

With reference to FIG. 3A, another aspect of the present disclosure comprises an apparatus for carrying out the thermochemical and photochemical reactions. In particular, the reactor 200 comprises a reaction vessel 210 having a vessel wall 212 defining a reaction chamber 214 and having one or more gas inlet(s) 216 configured for gaseous inflow of water and C₁ feedstock and a gas outlet 218 configured for gas outflow comprising hydrocarbons, both of which are in fluid communication with chamber 214. In some embodiments, a plurality of catalyst bodies 230 can sufficiently fill reaction chamber 214 to form a “packed bed.” In other embodiments, a catalytic body can comprise a corrugated surface or a porous or web-like body coated with the described catalyst. Reaction vessel 210 can further comprise a filter (not shown) at gas outlet 218 to prevent escape of catalyst bodies 130.

During use, the reaction vessel 210 can be exposed to solar radiation and heated at or above the boiling temperature of water to convert the gaseous mixture of water and C₁ feedstock into hydrocarbons including alkanes or alcohols having at least 2 carbons. Examples of the hydrocarbons that can be formed include methane, ethane, propane, butane, pentane, hexane, septane, octane, nonane, decane, methanol, ethanol, propanol, isopropanol, butanol, hexanol, acetic acid, acetone, alkyl benzene and oxygenates thereof, as well as longer alkanes, alcohols, and/or organic acids, or mixtures thereof. In some embodiments, reactor 200 can generate hydrocarbons having at least 2 carbons at a rate of at least 50 μg/g of catalyst per hour, 60 μg/g of catalyst per hour, 70 μg/g of catalyst per hour, 80 μg/g of catalyst per hour, 90 μg/g of catalyst per hour, 100 μg/g of catalyst per hour, 150 μg/g of catalyst per hour, 200 μg/g of catalyst per hour, 250 μg/g of catalyst per hour, 300 μg/g of catalyst per hour, 350 μg/g of catalyst per hour, or more. For example, as can be discerned from Table 3 in Example 4 below, a reactor in accordance with the present disclosure was shown to generate hydrocarbons having at least 2 carbons at a rate of approximately 87 μg/g of catalyst per hour (at 2.7 atm and 0.6 P_{w/c}, and when including CO, methane and methanol in this calculation, the productivity value of the catalyst is even greater, such as at 121 μg/g of catalyst per hour.

In some embodiments, the process conditions of the reactor can be adapted to generate one or more alkybenzene derivatives including toluene (C₇H₇), ethylbenzene (C₈H₁₀), propylbenzene (C₉H₁₂), ortho-, meta-, and para-xylenes (C₈H₁₀), ortho-, meta-, and para-methylethylbenzene (C₉H₁₂), ortho-, meta-, and para-methylpropylbenzene (C₁₀H₁₄), ortho-, meta-, and para-diethylbenzene (C₁₀H₁₄) as well as their oxygenates. For example, the process conditions can comprise a P_{w/c} between 0.2 and 1, such as 0.9, 0.8, 0.7, 0.6, 0.5, 0.4, or 0.3. In various embodiments, the process conditions are adapted such that alkyne cyclotrimerization reactions occur in the reactor in addition to the Fischer-Tropsch reactions.

Reaction vessel 210 is configured to operate at temperatures greater than 100° C. and to permit electromagnetic radiation, e.g., sun light, to pass through at least a section of vessel wall 212 and into chamber 214 where a plurality of catalytic bodies 130 are disposed. For example, vessel wall 212 can be composed of a substantially transparent material that is substantially heat tolerant and substantially UV tolerant material. In addition, in some embodiments, vessel wall 212 material may be required to withstand higher pressures, e.g., absolute pressures between 1 atm to 20 atm or any range therebetween. In some embodiments, vessel wall 212 can have a thickness less than about 10 mm, 5 mm, 4 mm, 3 mm, 2 mm, 1 mm, or any amount therebetween. In some embodiments, vessel wall 212 comprises any material through which radiation, such as sunlight, can pass through, and that can maintain high tensile strength at process temperatures, such as, temperatures up to 250° C., e.g., quartz, glass, (such as tempered glass and borosilicate glass), or the like.

One or more of walls 212 of the reaction vessel 210 or a portion thereof may be formed of transparent material. It is also possible that most or all of the walls 212 of reaction vessel 210 are transparent such that light may enter from many directions. For example, with reference to FIG. 3B, reaction vessel 210 may be a glass cylinder that is surrounded by an trough-like solar concentrator 206 that reflects light back into the reaction vessel. In another embodiment, reactor vessel 210 may have one side that is transparent to allow the incident radiation to enter and the other sides may have a reflective interior surface that reflects the majority of the solar radiation.

Reaction vessel 210 can be configured to operate at ambient operating pressures. Other embodiments, reaction vessel 210 can be configured to operate at much higher pressures to improve or vary hydrocarbon yields as appropriate. For example, operating pressures can be up to 30 atm. In some embodiments, reaction vessel 210 is configured to maintain an operating pressure of between about 1.0 atm and about 15 atm, or a smaller range therebetween. For example, operating pressures can be about 1 atm, 2 atm, 3 atm, 4 atm, 5 atm, 6 atm, 7 atm, 8 atm, 9 atm, 10 atm, 11 atm, 12 atm, 13 atm, and 14 atm, 15 atm, 16 atm, 17 atm, 18 atm, 19 atm, 20 atm, 21 atm, 22 atm, 23 atm, 24 atm, 25, atm, 26, atm, 27 atm, 28 atm, or 29 atm.

In some embodiments, reactor 200 can be heated largely if not entirely by solar energy. For example, again with reference to FIG. 3B, reactor 200 can be configured to receive solar radiation from a solar concentrator 206. Solar concentrator 206 comprises a reflective surface configured to direct solar radiation to reactor 200 and can be used to heat reactor 200 to a reaction temperature of 100° C., 110° C., 120° C., 130° C., 140° C., 150° C., 160° C., 165° C., 170° C., 175° C., 180° C., 185° C., 190° C., 195° C., 200° C., 205° C., 210° C., 215° C., 220° C., 225° C., 230° C., 240° C., 250° C., 260° C., 270° C., 280° C., 290° C., 300° C., or any range thereof or value therebetween. In the same or different embodiments, reactor 200 can be heated by a heater 270 to the desired reaction temperature. In some embodiments, reactor 200 may comprise radially extending conductive fins to distribute heat in reactor 200. Reactor 200 may also comprise a thermocouple 222 to monitor the temperature. Heater 270 can be used to regulate the reaction temperature as needed.

In some embodiments, heat from reactor 200 can be used to change water in liquid form to vapor form in a vaporization unit, further described below. As such, a heat exchanger (not shown) containing a heat transfer fluid can be disposed within reactor 200 to absorb some of the thermal energy provided by the sun or from the ongoing redox reactions and a conduit can transport the heated transfer fluid to the vaporization unit also comprising a heat exchanger to transfer the heat from the fluid to the water in the vaporization unit to convert the water feedstock to vapor. Moreover, heat transfer fluid can be used to facilitate regulation of the reaction temperature within reaction chamber 214.

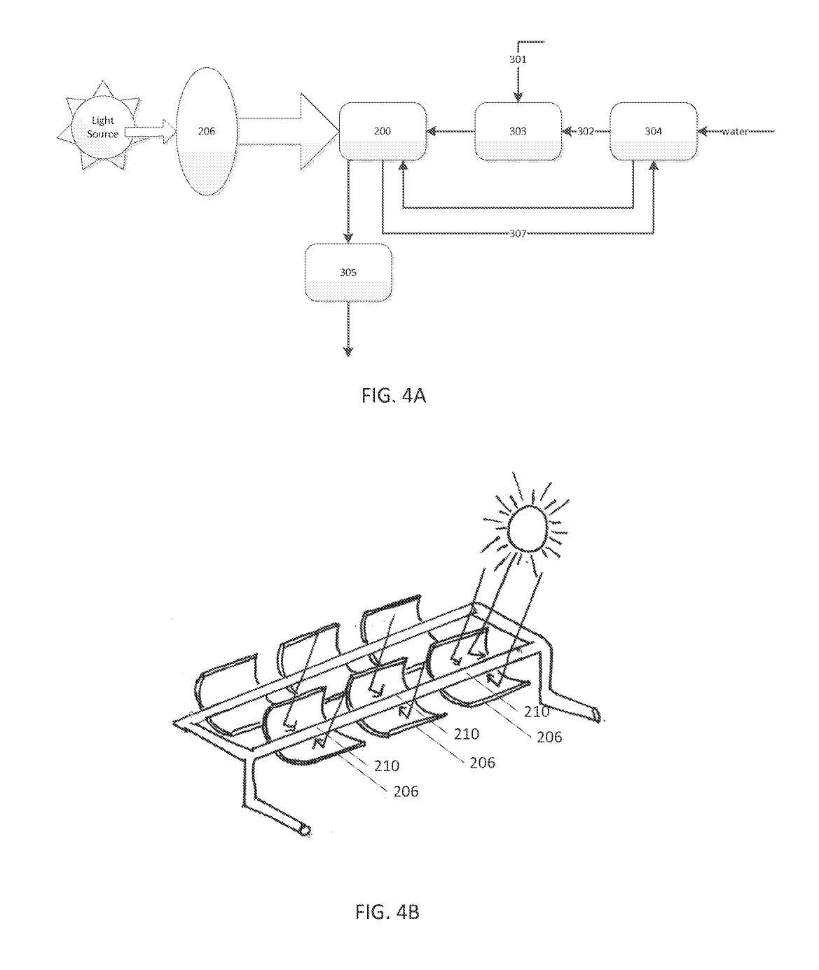

With reference to FIG. 4A, another aspect of the present invention comprises a system in which the above described reactor 200 is incorporated to generate hydrocarbons and separate the generated hydrocarbons from the gas outflow. A system can also comprise the described reactor 200 comprising an array of reaction vessels 210, as shown in FIG. 4B.

In order to convert a gaseous mixture of C₁ feedstock and water to hydrocarbons, gaseous feedstock of C₁ feedstock and water flows into the reaction chamber of reactor 200 containing the described catalyst. In some embodiments, the molar flow ratio of the water to C₁ feedstock is between 0.1 to 10.0, and such as between 0.1 and 3.0 or 0.1 and 4.0. In some embodiments, within the reaction chamber, the partial pressure ratio of water to C1 feedstock (P_{w/c}) can be maintained approximately at a value between 0.1 to 3, such as 0.2, 0.3, 0.4, 0.5, 0.6, 0.7, 0.8, 0.9, 1.0, 1.1, 1.2, 1.4, 1.6, 1.8, 2.0, 2.2, 2.4, 2.6, 2.8, or any value or range therebetween. The reaction chamber can be heated to or maintained at a desired reaction temperature and configured such that the described catalyst is exposed to solar radiation while the gaseous feedstock mixture is flowing there-through, thereby causing reactions that generate hydrocarbons from the C₁ feedstock and water.

When providing a flow of reactants into reactor 200, C₁ feedstock and the water in vapor form can flow into the reaction chamber as a mixture or as discrete inflows. System 300 can comprise a supply conduit 301 for providing C₁ feedstock. C₁ supply conduit 301 can merge with the water vapor supply conduit 302 to mix the two components at the desired ratios. In some embodiments, system 300 can comprise a gas proportioner or mixer 303 to facilitate mixing the gaseous components at the desired ratio. In some embodiments, the flow rate of each can be adjusted to control the relative ratio of the two components.

In order to provide water in vapor form, system 300 can also comprise a vaporization unit 304 configured to convert liquid water to steam. The steam generated flows from vaporization unit 304 into water vapor supply conduit 302. In some embodiments, vaporization unit 304 can comprise a heat exchanger through which a heat transfer fluid can flow. In some embodiments, the heat transfer fluid can flow from reactor 200 through the heat exchanger via conduit loop 307 to heat a surrounding bath of water. In the same or different embodiments, vaporization unit 304 can comprise, a mister, a humidifier, such as a evaporative humidifier, a natural humidifier, an impeller humidifier, a ultrasonic humidifier or a forced air humidifier, a vaporizer, or any other suitable device. In some embodiments, vaporization unit 304 also operates as a mixer or proportioner 303 such that C₁ feedstock can flow into vaporization unit 304 and mix with water vapor.

In order to extract the generated hydrocarbons, system 300 can further comprise separation device 305 for extracting a substantial portion of the hydrocarbons from the gaseous outflow. For example, separation device 305 can comprise at least one of a condensation column, membrane, centrifuge, an adsorbent material, or some combination thereof. While not shown in the figure, it is understood that in certain embodiments, once the hydrocarbons are extracted, the gaseous outflow may be recycled back to reactor 200.

In order to reduce or substantially remove unwanted products from the outflow, system 300 can further comprise another separation device (not shown). For example, dioxygen can be separated by passing the outflow through the separation device, such as at least one of a condensation column, an adsorbent material, membrane, or centrifuge. This separation device can intercept the outflow before or after it passes through to separation device 305. Once removed, in certain embodiments, the outflow can be recycled from the separation device into the reaction chamber.

To facilitate heating reaction chamber and to enhance the efficiency of the described catalyst, system 300 can comprise a solar concentrator 206 comprising a reflective surface(s) that directs sunlight to one or more reaction vessels 210. As shown in FIG. 4B, a system can also comprise a plurality of solar concentrators 206 and a plurality of reaction vessels 210. Reaction vessels 210 can be in fluid communication with each other or isolated therefrom. Reaction vessels can be configured so that the outflow from each flows into a single separation device 305.

In some embodiments, heating the reaction chamber can be caused by directing solar radiation from solar concentrator 306 to the reaction chamber. Alternatively or in addition thereto, a heater can be used to heat the reaction chamber. In addition, a heat exchanger can be located in reaction chamber facilitating the transfer of heat from chamber to a heat transfer fluid or vice versa.

### D. EXAMPLES

The present invention will be described in greater detail by way of specific examples. The following examples are offered for illustrative purposes only, and are not intended to limit the invention in any manner. Those of skill in the art will readily recognize a variety of noncritical parameters that can be changed or modified to yield essentially the same results.

### Example 1

### Preparation of Titanium Dioxide/Cobalt Catalyst

Titanium dioxide-cobalt catalyst were prepared by incipient wetness impregnation of TiO₂ (rutile) with sufficient aqueous solution of CoNO₃ (Alfa Aesar) to give a loading of 5% by mass cobalt when dried, calcined, and reduced. The impregnated TiO₂ was dried at room temperature for overnight and calcinations under air at 225° C. for 3 h and then sieved using No 100 (opening 0.15 mm). The dried catalyst was reduced at 400° C. in a flow of H₂ for 8 h. XPS spectroscopy indicated that only 1% of the cobalt present was in the metallic state, the remainder was present as Co₂O₃.

### Example 2

### Preparation of the Catalyst on a Substrate

The catalysis supports were Pyrex glass pellets having a 2 mm diameter. Before Co—TiO₂ catalyst was immobilized on the Pyrex glass pellets, these glass pellets were etched in 5M NaOH solution for 24 h at 70° C. After they had been rinsed with DI water, the glass pellets were soaked in an aqueous suspension, which was prepared with 3 g of catalyst as prepared in Example 1 and dispersed in 3.0 mL of DI water with the aid of an ultrasonic bath to which 3.0 mL of 5% w/w Nafion PTFE was added. After removing from the Catalyst-PTFE solution, the glass pellets were heated at 70° C. in a vacuum oven. The resulting pellets were opaque with a dull gray powder thinly coated on the surface.

### Example 3

### Preparation of Packed-Bed Thermophotocatalytic Reactor

A quartz tube having a length of 10 in. and a diameter of 1.4375 in. and a wall thickness of ⅛ in. and two plastic caps that fit on each end of the tube comprised the catalytic chamber. A stainless steel tube with an inner diameter of 0.25 in. and a length of 10 in. was placed along the center of the center of the quartz tube, and a cartridge heater was placed inside the stainless steel tube. The quartz tube was filled with the catalytic pellets as prepared in Example 2. Three holes were drilled on one of the caps and one hole was drilled in the other. Graphite tape, metal camps, and high temperature PTFE O-rings were placed between the caps and the tube to provide the necessary seal. A thermocouple was inserted into one hole, the cartridge heater was inserted through a central hole, and a fitting for the inflow gas line was placed in the third. A fitting for the outflow gas line was placed in the hole of the other cap. The CO₂ gas was regulated by a digital flow meter and directed into a water saturation unit that humidified the gas. The cartridge heater was controlled by a discrete feedback controller to maintain the desired reaction temperature as measured by the thermocouple. The quartz tube was surrounded by four Hg UV producing lamps with a total power of 850 W. A schematic is shown in FIG. 4.

### Example 4

### Conversion of Carbon Dioxide and Water into Hydrocarbons Using the Catalyst

The system as described in Example 3 was used to study catalyst performance and carbon products produced under various process conditions.

In a first study, the reaction was run under 1 atmosphere pressure for 8 hours. Carbon dioxide flowed into the saturator having 20 mL of water to mix the carbon dioxide with water vapor. The temperature of the saturator was set to produce the desired flow rate of water vapor. The input of carbon dioxide was set at the desired flow rate of 50 mL/min at 0 psig. The water flow rate was 0.03 mL/min. This corresponds to a CO₂:H₂O molar ratio of 1:3. Many runs were conducted at six different reactor temperatures: 110° C., 130° C., 150° C., 180° C., 200° C., and 220° C. Two phase of TiO₂ were tried, rutile and anatase.

Liquid aliquots were collected and tested on a Shimadzu GC-MS-2010SE chromatograph coupled with a MS QP2010 detector and a AOC-4 20S sampler. The column was a Shimadzu SHRX105MS (30-m length and 0.25-mm inner diameter, part #220-94764-02) set at 45° C. for 5 minutes then increased to 150° C. at a rate of 10° C./min. The MS detector was set at 250° C., and helium was used as the carrier gas. A 1 μL sample of the liquid aliquot was injected into the GC-MS. The results are provided in Table 1 below.

TABLE 1
Effect of temperature and TiO₂ phase on products
at 1 atm pressure and P_{w/c} = 0.6.

| | Phase and | μg/g_{cat} · h | | | | |
|---|---|---|---|---|---|---|
| | Temperature | | | | Acetic | C8H10 |
| No. | (° C.) | MeOH | EtOH | IPA | acid | product |
| 1 | TiO₂(rutile)-110 | 0.30 | — | — | — | — |
| 2 | TiO₂(rutile)-130 | 3.00 | — | 0.60 | — | −0.0024 |
| 3 | TiO₂(rutile)-150 | 2.70 | 0.38 | — | — | — |
| 4 | TiO₂(rutile)-180 | 0.30 | 0.24 | — | — | 0.0024 |
| 5 | TiO₂(rutile)-200 | 0.23 | 0.11 | — | — | 0.016 |
| 6 | TiO₂(rutile)-220 | — | — | — | — | 0.0022 |
| 7 | dark-CO₂-200 C. | — | — | — | — | — |
| 8 | Light-N₂-200 C. | — | — | — | — | — |
| 9 | TiO₂(rutile) | — | — | — | — | — |
| | only-200 C. | | | | | |
| 10 | TiO₂(anatase)-110 | — | 4.6 | — | — | 3.4E−4 |
| 11 | TiO₂(anatase)-130 | 25.8 | — | — | — | 2.4E−4 |
| 12 | TiO₂(anatase)-150 | 2.70 | — | 1.20 | — | 4.7E−4 |
| 13 | TiO₂(anatase)-180 | — | 5.5 | 4.2 | 16.2 | 7.3E−4 |
| 14 | TiO₂(anatase)-200 | — | 130 | 92.4 | 90.6 | 3.6E−3 |

A second study was also conducted in a similar manner with the set up as described in Example 3. A titanium dioxide-cobalt catalyst was prepared by wet impregnation as described in Example 1, except that the anatase form of TiO₂ was used for this study.

For the runs conducted, carbon dioxide flowed into the saturator having 20 mL of water to mix the carbon dioxide with water vapor. The temperature of the saturator was set to produce the desired flow rate of water vapor. The input of carbon dioxide was set at the desired flow rate of 50 mL/min at 0 psig. The water flow rate was 0.03 mL/min. This corresponds to a CO₂:H₂O molar ratio of 1:3. The reaction temperature, the reaction pressure, and the partial pressure ratios of the reactants, water and CO₂ were varied for purposes of this study.

For most runs, to determine the amount and type of products in the gaseous effluent, the effluent was passed through an online-reactor gas analyzer by Custom Solutions Group (CSG), Houston, Tex. The gas analyzed is built on a Shimadzu Model GC-2014 and equipped with a split/splitless injection port, a three channel automated pressure control and auto flow control, and TCD and FID detectors. The instrument was precalibrated by CSG for analysis of light to medium hydrocarbons and their oxygenates, CO, CO₂, O₂, H₂, and N₂.

The permutations of pressure, temperature, and partial pressure ratio that were studied are summarized in Tables 2 and 3 alongside the results of those runs. Each run was conducted for 8 hours. Results for the runs conducted at 200 C are provided in Tables 3 and 4.

TABLE 2
Effect of temperature, pressure, and the partial pressure ratio on product make-up

| | | T | P | | | |
|---|---|---|---|---|---|---|
| Run | Catalyst | (C.) | (atm) | PH₂O/PCO₂ | Irrad | Products |
| 1 | Co/TiO₂ | 110 | 1.0 | 1.1 | + | CH₃OH |
| 2 | Co/TiO₂ | 130 | 1.0 | 1.1 | + | CH₃OH |
| 3 | Co/TiO₂ | 150 | 1.0 | 1.1 | + | CH₃OH |
| 4 | Co/TiO₂ | 180 | 1.0 | 1.1 | + | CH₃OH, C₃H₇OH |
| 5 | Co/TiO₂ | 200 | 1.0 | 1.1 | + | CH₃OH, C₃H₇OH |
| 6 | Co/TiO₂ | 220 | 1.0 | 1.1 | + | none |
| 7 | Co/TiO₂ | 200 | 1.0 | 1.1 | − | none |
| 8 | Co/TiO₂ | 200 | 1.0 | no CO₂ | + | none |
| 9 | TiO₂ | 200 | 1.0 | 1.1 | + | none |
| 10 | Co/TiO₂ | 200 | 1.0 | 1.1 | + | |
| 11 | Co/TiO₂ | 200 | 1.0 | 0.6 | + | CH₃OH, C₂H₅OH, |
| | | | | | | C₃H₇OH, C₃H₆O, |
| | | | | | | CH₃COOH |
| 12 | Co/TiO₂ | 200 | 1.0 | 0.6 | − | none |
| 13 | Co/TiO₂ | 200 | 2.7 | 0.6 | | CH₃OH, C₂H₅OH, |
| | | | | | + | C₃H₇OH, CH₃COOH, |
| | | | | | | C₃H₆O, C₄H₉OH, C₆H₁₂O, |
| | | | | | | C₈H₁₀, C₉H₁₂, C₁₀H₁₄ |
| 14 | Co/TiO₂ | 200 | 2.7 | 0.6 | − | none |
| 15 | CoO/TiO₂ | 200 | 2.7 | 0.6 | | undetermined |
| 16 | Co/TiO₂ | 200 | 2.7 | 0.6 (D₂O) | | deuterium incorporated |
| | | | | | | into CH₃OH, C₂H₅OH, |
| | | | | | | C₃H₇OH, CH₃COOH, |
| | | | | | | C₃H₆O, C₄H₉OH, C₆H₁₂O, |
| | | | | | | C₈H₁₀, C₉H₁₂, C₁₀H₁₄ |
| 17 | Co/TiO₂ | 200 | 2.7 | 0.6 (¹³CO₂) | | 13-carbon incorporated |
| | | | | | | into CH₃OH, C₂H₅OH, |
| | | | | | | C₃H₇OH, CH₃COOH, |
| | | | | | | C₃H₆O, C₄H₉OH, C₆H₁₂O, |
| | | | | | | C₈H₁₀, C₉H₁₂, C₁₀H₁₄ |

As gleaned from the results in Table 2, methanol was observed at the lower temperatures (i.e., 110 to 150 C), but higher Cₙ products (>C1) began to appear at temperatures of 180 C or higher, predominantly as iso-propanol (Run 4), and increased upon going to 200 C (Run 5) and 220 C (Run 6) with an apparent yield maximum at 200 C. Lowering the P_{w/c} from 1.2 to 0.6 resulted in an increase in the number of products obtained to include ethanol, acetic acid, isopropanol, and acetone (Run 7). The most striking result was obtained with the application of 2.7 atm of pressure at 200 C (P_{w/c}=0.6) as seen in Run 11. Now in addition to the C1-3 products, hydrocarbons with Cₙ of 4, 6, 8, 9 and 10 were also obtained, with the last three (C8-10) being pure hydrocarbons. Control reactions have established that light, TiO₂, Co, CO₂, and elevated temperature (180-200 C) are all required.

In specific runs, isotopically labelled reactants, 30% enriched ¹³CO₂ (Run 8) or 99% enriched D₂O (Run 9) or were used to establish that H₂O and CO₂ where the sources for hydrogen and carbon in the products, respectively. In both cases, the organic products showed the expected incorporation of the label as determined by GC-MS (see supporting information). The 13-carbon label appearing in the relative amount expected statistically for a 30% enriched feedstock. Deuterium incorporation was lower than expected for a 99% enriched feedstock but still the dominant isotope of hydrogen found in the product (i.e. the formation of products such as C₈D₈H₂). The non-statistical level of H over D incorporation is likely due to kinetic isotope effects, and the presence of surface bound H₂O in the reactor and catalyst despite an initial purge with CO₂.

TABLE 3
Effect of pressure and water/CO₂ partial pressure ratio (P_{w/c}) on product yield at 200 C.

| | | Productivity (μγ/gh) | | | | | | Productivity (μμoλ e/gh) | | | | | |
|---|---|---|---|---|---|---|---|---|---|---|---|---|---|
| | | Pressure (atm) | | | | | | | | | | | |
| | | 1 | | 2.7 | | 6.1 | | 1 | | 2.7 | | 6.1 | |
| Products | | Pw_{/c} | | | | | | | | | | | |
| Cn | Formula | 0.6 | 1.2 | 0.6 | 1.2 | 0.4 | 1.2 | 0.6 | 1.2 | 0.6 | 1.2 | 0.4 | 1.2 |
| | O2 | 453 | 210 | 189 | 140 | 230 | 306 | 56.7 | 26.3 | 23.6 | 17.5 | 28.8 | 38.3 |
| | H2 | 2.4 | 4.1 | 8.4 | 17.2 | 5.5 | 9.8 | 2.4 | 4.1 | 8.4 | 17.2 | 5.5 | 9.8 |
| C1 | CO | 44.0 | 45.1 | 33.1 | 23.0 | 25.3 | 38.4 | 3.1 | 3.2 | 2.4 | 1.6 | 1.8 | 2.7 |
| | CH4 | 0.9 | 0.7 | 0.9 | 0.9 | 1.9 | 0.6 | 0.4 | 0.3 | 0.5 | 0.4 | 0.9 | 0.3 |
| | CH2O2 | | | | 4.7 | 42.8 | 7.4 | | | | 0.2 | 1.9 | 0.3 |
| | CH3OH | 0.2 | | 0.2 | | 1.4 | 4.6 | | | | | 0.3 | 0.9 |
| C2 | C2H4 | 0.4 | 0.1 | 1.7 | 0.3 | | 0.6 | 0.2 | 0.1 | 0.7 | 0.1 | | 0.2 |
| | C2H6 | 3.4 | 4.0 | 1.6 | 3.0 | 2.2 | 3.5 | 1.6 | 1.9 | 0.7 | 1.4 | 1.0 | 1.6 |
| | C2H6O | 1.6 | 0.1 | 0.1 | | 0.7 | | 0.4 | | | | 0.2 | |
| | C2H4O2 | 8.6 | 9.0 | 44.4 | 3.4 | 13.8 | 36.7 | 1.1 | 1.2 | 5.9 | 0.4 | 1.8 | 4.9 |
| C3 | C3H6 | 0.6 | 0.3 | | 1.5 | 1.6 | 0.4 | 0.3 | 0.1 | | 0.6 | 0.7 | 0.2 |
| | C3H8 | 1.0 | 1.1 | 1.2 | 1.1 | 1.7 | 1.1 | 0.5 | 0.5 | 0.5 | 0.5 | 0.8 | 0.5 |
| | C3H8O | 0.2 | 0.1 | 0.2 | 2.5 | 0.5 | 5.7 | 0.1 | | 0.1 | 0.8 | 0.2 | 1.7 |
| | C3H8O | | | | | | 10.2 | | | | | | 3.1 |
| | C3H4O3 | | | | | 0.2 | 0.0 | | | | | | |
| C4 | C4H8 | 1.1 | | 4.8 | | | 1.2 | 0.5 | | 2.1 | | | 0.5 |
| | C4H10 | | 0.1 | | | 4.8 | 0.0 | | 0.1 | 0.0 | | 2.1 | 0.0 |
| | C4H10O | | | 28.4 | | 1.7 | 2.1 | | | 9.2 | | 0.5 | 0.7 |
| | C4H8O2 | | | | | 3.8 | 1.4 | | | | | 0.9 | 0.3 |
| C5 | C5H12 | | | | | 2.2 | 0.2 | | | | | 1.0 | 0.1 |
| | C5H12O2 | | | | | 7.4 | 0.0 | | | | | 1.8 | |
| C7 | C7H6O2 | | | | | 4.3 | 0.0 | | | | | 1.1 | |
| C8 | C8H10 | | | 0.5 | | | 0.0 | | | 0.2 | | 0.0 | |
| | C8H16O5 | | | | | 6.7 | 0.0 | | | | | 1.3 | |
| C9 | C9H12 | | | 4.3 | | | 0.0 | | | 1.7 | | | |
| | C10H14 | | | | | | 0.0 | | | | | | |
| C10 | C10H12O2 | | | | | 10.5 | 0.0 | | | | | 3.1 | |
| | H2 | 2.4 | 4.1 | 8.4 | 17.2 | 5.5 | 9.8 | 2.4 | 4.1 | 8.4 | 17.2 | 5.5 | 9.8 |
| | C2+ | 16.9 | 14.8 | 87.2 | 11.8 | 62.0 | 63.0 | 4.6 | 3.8 | 21.2 | 3.9 | 16.5 | 13.8 |
| | C1-4 | 61.9 | 60.6 | 116.6 | 40.4 | 102.3 | 113.8 | 8.2 | 7.4 | 22.1 | 6.2 | 13.1 | 17.9 |
| | C5+ | 0.0 | 0.0 | 4.8 | 0.0 | 31.1 | 0.2 | 0.0 | 0.0 | 1.9 | 0.0 | 8.3 | 0.1 |
| | Sum | 81.3 | 79.6 | 217.0 | 69.3 | 201.0 | 186.8 | 10.6 | 11.5 | 32.4 | 23.3 | 26.9 | 27.8 |
| | O2 Yld(%) | | | | | | | 535 | 229 | 73 | 75 | 107 | 138 |
| | IPQY(%) | | | | | | | 0.06 | 0.07 | 0.19 | 0.13 | 0.15 | 0.16 |

TABLE 4
Product Distribution by Carbon Number (Cn), Total Pressure
and Partial Pressure Ratio of Water to CO₂.

| | Hydrocarbon Productivity | | | | | | Hydrocarbon Molar Productivity | | | | | |
|---|---|---|---|---|---|---|---|---|---|---|---|---|
| | (μγ/gh) | | | | | | (μμoλ e-/gh) | | | | | |
| | Pressure (atm) | | | | | | | | | | | |
| | 1 | | 2.7 | | 6.1 | | 1 | | 2.7 | | 6.1 | |
| | Pw_{/c} | | | | | | | | | | | |
| | 0.6 | 1.2 | 0.6 | 1.2 | 0.4 | 1.2 | 0.6 | 1.2 | 0.6 | 1.2 | 0.4 | 1.2 |
| C1 | 45.0 | 45.8 | 34.2 | 28.6 | 71.4 | 51.0 | 3.6 | 3.6 | 2.8 | 2.3 | 4.9 | 4.3 |
| C2 | 14.0 | 13.3 | 47.7 | 6.7 | 16.8 | 40.8 | 3.3 | 3.1 | 7.4 | 2.0 | 3.1 | 6.8 |
| C3 | 1.8 | 1.4 | 1.4 | 5.1 | 3.9 | 17.3 | 0.8 | 0.6 | 0.6 | 1.9 | 1.6 | 5.4 |
| C4 | 0 | 1.1 | 0.1 | 33.2 | 0.0 | 10.2 | 0.5 | 0.1 | 11.3 | 0.0 | 3.5 | 1.5 |
| C5 | 0 | 0.0 | 0.0 | 4.8 | 0.0 | 31.1 | 0.0 | 0.0 | 1.9 | 0.0 | 8.3 | 0.1 |
| Sum | 60.9 | 61.5 | 83.5 | 78.5 | 92.1 | 150.4 | 8.2 | 7.4 | 24.0 | 6.2 | 21.3 | 18.0 |

In this second study, product carbon number (CO distribution and incident photon quantum yields (IPQYs) show a strong dependence on the reaction pressure, temperature, irradiation levels, and the P_{H2O}/P_{CO2} ratio (P_{w/c}), suggesting that the photochemical steps are not rate determining here. For example, at 200 C, an increase in pressure from 1 atm to 6.1 atm increased the average productivity increased from 80 to 200 μg/gh (units: μg fuel/g_{catalyst}h), respectively, an overall increase of 250% and shifts the product distribution to higher molecular weight products. The products and mass yields obtained in this latter run (200 C, 6.1 atm, P_{w/c} 0.6) are H₂ (6.5%), CO (25.5%), CH₄ (0.7%), CH₃OH (0.1%), C₂H₄ (1.3%), C₂H₆ (1.2%), H₃C₂O₂H (34.2%), C₃H₈ (0.9%), C₃H₇OH (0.2%), C₄H₈ (3.7%), C₄H₁₀ (21.9%), C₈H₁₀ (0.4%), and C₉H₁₂ (3.3, of which 64% are liquid products.

O₂ was also isolated in a 2 to 5-fold stoichiometric excess compared to the reduced product obtained at 1 atm. At higher pressures, the O₂ yield was either near stoichiometric (˜75% for the runs at 2.6 atm) or only present in modest excess (107-138% for the runs at 6.1 atm). As the products should be present stoichiometrically, these data suggest we have not accounted for all the reduction products in certain runs. For runs at 1 atm, these are likely to be high boiling point oxygenates adsorbed onto the catalyst or surface of the reactor, especially near the exit zone at which the temperature drops considerably. At 6.1 atm, the missing product could be either oxygenates like above or heavy hydrocarbons which condense in the exit zone or transport tubes. Lastly, dioxygen plus both components of syngas, CO and H₂, are observed as co-products in the studied reactor, so it seems reasonable that a water splitting reaction and a reverse water gas shift reaction are functional, but it may be that most of the H₂ and CO are not released from the cobalt surface.

The presence of an excess or near stoichiometric amount of O₂ suggests that the back reaction, O₂ oxidation of H₂ or hydrocarbon products, is somewhat inhibited, most likely due to the low O₂ concentration, estimated to be between 4% and 0.4% v/v in any given run. One explanation for the large excess of O₂ seen at 1 atm, but not at 2.6 or 6.1 atm, is that the space velocity is faster at lower pressures, meaning the O₂ is swept from the reaction chamber more quickly and has less time to participate in the back reaction. As such, mass flow rates and space velocity can be adjusted to remove O₂ more quickly from the reactor so it can be separated from the flow.

As mentioned, CO and H₂ are both observed as products, yet both are reactants for the Fischer-Tropsch reaction. Also mentioned, the data suggests that not all of the CO or H₂ equivalents (i.e. surface cobalt hydrides) are released in the gas phase but instead are generated on the surface of the cobalt islands and consumed immediately in subsequent chain-forming reactions. The reasoning here is similar to the poor O₂ back reaction rates, even with 100% release into the gas phase, the resulting low partial pressures of CO and H₂ would make it very unlikely that a chain-forming reaction mechanism could be sustained. In some embodiments, these flow with these products and can be recycled into the reactor chamber to further favor CO₂ reduction and Fischer-Tropsch type reactions.

The presence of alyklbenzene products reveals that one of the chain-forming reactions is likely proceeding via the formation of alkyl alkynes and subsequent alkyne trimerization. While higher hydrogen yields may be anticipated with more water, the better selectivity towards higher Cn products at P_{w/c} of 0.6 is, in part, a reflection of an unusual synthetic pathway that appears to be operational at this lower water partial pressure. All of the products with Cₙ>6 are all identified as variously substituted alkylbenzenes or oxygenates thereof, which is atypical of traditional FTS product distributions.

Currently, the highest IPQY obtained is 0.19% on a per electron stored basis (or 0.105% on a H₂ equivalent basis), but this is a reflection of the early stage of this work rather than any practical limitation. There is a significant (2 to 3-fold) jump in ICPY upon increasing the pressure from 1 atm to 2.6 atm, but little further change upon increasing the pressure to 6.1 atm. In theory, quantum yields of 30-50% at 200 C are possible and if the TiO₂ could be replaced by a semiconductor absorber that covered more of the visible spectrum (i.e. <700 nm), then overall solar to fuel (STF) conversion efficiencies of 5-15% are reasonable goals. However, the process in the study is not optimized and these initial studies indicate that higher yields and/or higher order hydrocarbons are accessible at higher pressures, higher temperatures, and other P_{w/c} ratios.

The above specification and examples provide a complete description of the structure and use of an exemplary embodiment. Although certain embodiments have been described above with a certain degree of particularity, or with reference to one or more individual embodiments, those skilled in the art could make numerous alterations to the disclosed embodiments without departing from the scope of this invention. As such, the illustrative embodiments of the present photothermocatalytic compositions, reactors, systems, and process are not intended to be limited to the particular forms disclosed. Rather, they include all modifications and alternatives falling within the scope of the claims, and embodiments other than the one shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not to be interpreted as including means-plus- or step-plus-function limitations, unless such a limitation is explicitly recited in a given claim using the phrase(s) “means for” or “step for,” respectively.

1. A method of converting a gaseous mixture comprising water and at least one of CO and CO₂ to hydrocarbons, the method comprising:
providing a flow of water and at least one of CO and CO₂ into a reaction chamber containing a supported metal catalyst;
heating the reaction chamber to a reaction temperature greater than 100° C.; and
exposing the supported metal catalyst to electromagnetic radiation,
thereby causing a reaction that generates hydrocarbons from the provided flow,
wherein the supported metal catalyst comprises a photoactive material support and a plurality of conductive particles disposed on the support.

2. The method of claim 1, wherein the reaction temperature is between 100° C. and 300° C.

3. (canceled)

4. The method of claim 1, wherein heating the reaction chamber comprises directing sunlight reflecting from a solar concentrator onto the reaction chamber.

5. The method of claim 1, wherein the photoactive material support is a semiconductor support and the supported metal catalyst is the semiconductor support having a surface with metal particles interspersed on the surface.

6. The method of claim 5, wherein the semiconductor support comprises a metal oxide and the metal particles comprise a metal selected from Fe, Co, Ni, Cu, Ru, Rh, Ir, Pd, Pt, and Ag or any combination thereof.

7. (canceled)

8. (canceled)

9. The method of claim 5, wherein the supported metal catalyst is modified by addition of a hygroscopic additive.

10. The method of claim 9, wherein the hygroscopic additive comprises a salt comprising at least one of the following anions: PO₄³⁻, HPO₄²⁻, H₂PO⁴⁻, SO₄²⁻, HSO₄⁻, CO₃²⁻, OH⁻, F⁻, Cl⁻, Br⁻ and I⁻ and at least one of the following cations: Li⁺, Na⁺, K⁺, Rb⁺, Cs⁺, NH₄⁺, Be²⁺, Mg²⁺, Ca²⁺, Sr²⁺, Ba²⁺ and Al³⁺.

11. The method of claim 9, wherein the hygroscopic additive comprises an acid and wherein the acid comprises at least one of the following: H₂SO₄, H₃PO₄, HF, HCl, HBr, and HI.

12. The method of claim 9, wherein the hygroscopic additive is disposed on a surface of the semiconductor support.

13. The method of claim 5, wherein the supported metal catalyst is further modified by addition of a redox-active additive.

14. (canceled)

15. The method of claim 13, wherein the redox-active additive comprises a salt comprising at least one of the following cations: Mn²⁺, Mn³⁺, Mn⁴⁺, Fe²⁺, Fe³⁺, Co²⁺, Co³⁺, Ni²⁺, Ru²⁺, Ru³⁺, Rh⁴⁺, Rh⁺, Rh²⁺, Rh³⁺, Ir⁺, Ir²⁺, and Ir³⁺ and at least one of the following anions: PO₄³⁻, HPO₄²⁻, H₂PO₄⁻, SO₄²⁻, HSO₄⁻, CO₃²⁻, O²⁻, OH⁻, F⁻, Cl⁻, Br⁻ and I⁻.

16. The method of claim 13, wherein the supported metal catalyst is further modified by addition of a basic metal oxide promotor of the Fischer-Tropsch synthesis reaction.

17. The method of claim 16, wherein the basic metal oxide promotor comprises a oxide salt comprising at least one of the following cations: Sc³⁺, Y³⁺, La³⁺, Ce³⁺, Pr³⁺, Nd³⁺, Sm³⁺, Eu³⁺, Gd³⁺, Tb³⁺, Dy³⁺, Ho³⁺, Er³⁺, Tm³⁺, Yb³⁺, Ac³⁺, Th³⁺, Pa³⁺, and U³⁺.

18. The method of claim 13, wherein the redox-active additive is disposed on a surface of the semiconductor support.

19. (canceled)

20. (canceled)

21. The method of claim 20, wherein the pellet is optically transparent, thermally conductive, or both.

22-33. (canceled)

34. The method of claim 1, wherein the hydrocarbons include alkanes or oxygenates having at least 2 carbons.

35. (canceled)

36. The method of claim 1, wherein the hydrocarbons include alkylbenzenes or oxygenates thereof.

37. (canceled)

38. The method of claim 1, wherein the reactor conditions are adapted such that alkyne cyclotrimerization reactions occur therein to form substituted benzenes, especially at lower partial pressures of water.

39-42. (canceled)

42. The method of claim 1, wherein the supported metal catalyst absorbs electromagnetic radiation having wavelength between 200 nm and 700 nm.

43. The method of claim 1, wherein the hydrocarbons are produced at a rate of at least 100 μg/g of catalyst per hour.

44-74. (canceled)
